(19) United States
(12) Patent Application Publication
(10) Pub. No.: US 2025/0289786 A1
(43) Pub. Date: Sep. 18, 2025

(54) CHLORINATED MONOCARBONYL CURCUMIN COMPOUNDS, PREPARATION METHOD, AND APPLICATION

(71) Applicant: Southwest Medical University, Luzhou (CN)

(72) Inventors: Ling ZHAO, Luzhou (CN); Yumeng WEI, Luzhou (CN)

(73) Assignee: Southwest Medical University, Luzhou, (CN)

(21) Appl. No.: 19/062,067

(22) Filed: Feb. 25, 2025

(30) Foreign Application Priority Data
Mar. 14, 2024 (CN) 202410294173.2

(51) Int. Cl. C07D 213/68; A61K 31/45

(52) U.S. Cl. CPC C07D 213/68; A61K 31/45

(57) ABSTRACT

Chlorinated monocarbonyl curcumin compounds, a preparation method, and an application are provided. 3,5-dichloro-4-hydroxybenzaldehyde, 3-chloro-4-hydroxy-5-methoxybenzaldehyde, 4-piperidone, 1-methyl-4-piperidone, 1-ethyl-4-piperidone and 1-isopropyl-4-piperidone are used as raw materials, the unstable β-dicarbonyl structure of curcumin is substituted by monocarbonyl group, and chlorine substitution is carried out at the corresponding position of the benzene ring to obtain more stable monocarbonyl curcumin compounds CAK01-CAK07, which can improve the solubility and biological activity of curcumin at the same time, so that it has higher safety, better pharmacokinetic behavior and higher anti-inflammatory and antioxidant activity, the curcumin compounds are used to prepare drugs for the treatment of inflammation-related diseases.

### CROSS REFERENCE TO THE RELATED APPLICATIONS

This application is based upon and claims priority to Chinese Patent Application No. 202410294173.2, filed on Mar. 14, 2024, the entire contents of which are incorporated herein by reference.

### TECHNICAL FIELD

The invention relates to medical technology, particularly, chlorinated aminocarbonyl curcumin compounds, a preparation method, and an application.

### BACKGROUND

Natural drugs have the advantages of multiple targets and low toxicity and side effects, it is an important source of new drugs and provides an important idea for developing new structural drugs. Curcumin (CU) is the main bioactive polyphenol component in turmeric, and many studies have confirmed the antidepressant and anxiolytic effects of CU. However, the weak stability, poor solubility, and low bioavailability of CU severely limit its clinical application. The instability and irregular metabolism of CU are mainly caused by the β-dicarbonyl structural unit in its structure, the β-diketone group, methoxy group, and phenolic hydroxyl group in the structure are not only responsible for molecular pharmacological activity, but also important sites for structural modification. It was found that the methyl, methoxy, and chloro substituted analogues on the phenolic ring of CU had significantly enhanced anti-inflammatory and antioxidant activity. Dienone monocarbonyl curcumin analogues can overcome the instability and pharmacokinetic defects of CU. The monoketone monocarbonyl curcumin analogue sAc15 has better antioxidant and central protective effects, and shows the potential to treat cerebral ischemia-reperfusion damage in a mouse cerebral artery occlusion (MCAO) model. However, there are few reports on compounds that can significantly improve solubility and biological activity in curing a variety of inflammation-related diseases, including depression.

### SUMMARY

The purpose of this invention is to provide chlorinated monocarbonyl curcumin compounds with higher solubility and biological activity.

Another purpose of the invention is to provide a preparation method and an application of the chlorinated monocarbonyl curcumin compounds.

The chlorinated monocarbonyl curcumin compounds described in this invention are represented by the following structural formula:

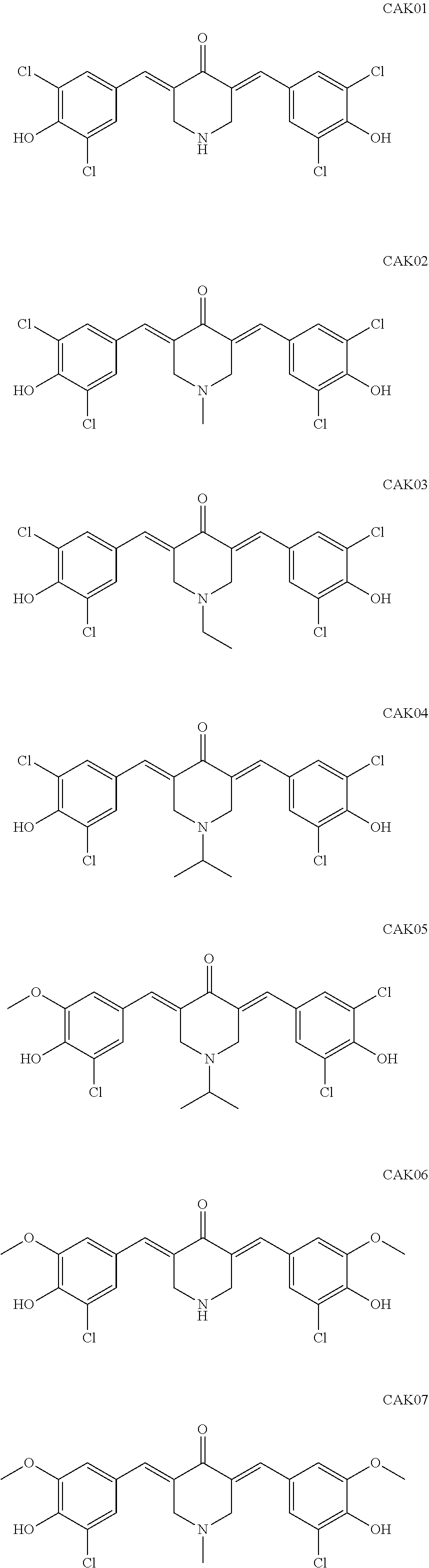

The preparation method for the chlorinated monocarbonyl curcumin compounds described in the invention is as follows:

Dissolving 2-10 parts of 3,5-dichloro-4-hydroxybenzaldehyde and 1-5 parts of 4-piperidone in 15-50 parts of saturated hydrogen chloride glacial acetic acid solution, and stirring at 22-28° C. for 12-24 h to obtain solution A, after standing for 12-24 h, filtering solution A to obtain a precipitate, washing the precipitate repeatedly with deionized water and anhydrous ethanol for 2-4 times, and drying the remaining precipitate to obtain compound CAK01.

Dissolving 2-10 parts of 3,5-dichloro-4-hydroxybenzaldehyde and 1-5 parts of 1-methyl-4-piperidone in 15-50 parts of saturated hydrogen chloride glacial acetic acid solution, and stirring at 22-28° C. for 12-24 h to obtain solution B, after standing for 12-24 h, filtering solution B to obtain a precipitate, washing the precipitate repeatedly with deionized water and anhydrous ethanol for 2-4 times, and drying the remaining precipitate to obtain compound CAK02.

Dissolving 2-10 parts of 3,5-dichloro-4-hydroxybenzaldehyde and 1-5 parts of 1-ethyl-4-piperidone in 15-50 parts of saturated hydrogen chloride glacial acetic acid solution, and stirring at 22-28° C. for 12-24 h to obtain solution C, after standing for 12-24 h, filtering solution C to obtain a precipitate, washing the precipitate repeatedly with deionized water and anhydrous ethanol for 2-4 times, and drying the remaining precipitate to obtain compound CAK03.

Dissolving 2-10 parts of 3,5-dichloro-4-hydroxybenzaldehyde and 1-5 parts of 1-isopropyl-4-piperidone in 15-50 parts of saturated hydrogen chloride glacial acetic acid solution, and stirring at 22-28° C. for 12-24 h to obtain solution D, after standing for 12-24 h, filtering solution D to obtain a precipitate, washing the precipitate repeatedly with deionized water and anhydrous ethanol for 2-4 times, and drying the remaining precipitate to obtain compound CAK04.

Dissolving 2-10 parts of 3-chloro-4-hydroxy-5-methoxybenzaldehyde and 1-5 parts of 1-isopropyl-4-piperidone in 15-50 parts of saturated hydrogen chloride glacial acetic acid solution, and stirring at 22-28° C. for 12-24 h to obtain solution E, after standing for 12-24 h, filtering solution E to obtain a precipitate, washing the precipitate repeatedly with deionized water and anhydrous ethanol for 2-4 times, and drying the remaining precipitate to obtain compound CAK05.

Dissolving 2-10 parts of 3-chloro-4-hydroxy-5-methoxybenzaldehyde and 1-5 parts of 4-piperidone in 15-50 parts of saturated hydrogen chloride glacial acetic acid solution, and stirring at 22-28° C. for 12-24 h to obtain solution F, after standing for 12-24 h, filtering solution F to obtain a precipitate, washing the precipitate repeatedly with deionized water and anhydrous ethanol for 2-4 times, and drying the remaining precipitate to obtain compound CAK06.

Dissolving 2-10 parts of 3-chloro-4-hydroxy-5-methoxybenzaldehyde and 1-5 parts of 1-methyl-4-piperidone in 15-50 parts of saturated hydrogen chloride glacial acetic acid solution, and stirring at 22-28° C. for 12-24 h to obtain solution G, after standing for 12-24 h, filtering solution G to obtain a precipitate, washing the precipitate repeatedly with deionized water and anhydrous ethanol for 2-4 times, and drying the remaining precipitate to obtain compound CAK07.

The application of chlorinated monocarbonyl curcumin compounds in the preparation of drugs for the treatment of inflammation-related diseases.

Inflammation-related diseases include depression, anxiety, schizophrenia, neuropathic pain, Alzheimer's disease, Parkinson's syndrome, glioma, lung cancer, liver cancer, breast cancer, colon cancer, rheumatoid arthritis, systemic lupus erythematosus, osteoarthritis, gouty arthritis, skin inflammation, autoimmune diseases, cardiovascular diseases, atherosclerosis, colorectal inflammation, allergic inflammation, hepatitis, and pneumonia.

By adopting the above technical scheme, the invention has the following beneficial effects:

The invention uses curcumin and its piperidine monocarbonyl curcumin analogues as lead compounds, designs and synthesizes a series of new chlorinated monocarbonyl curcumin compounds, and explores their structure-activity relationship, which provides more potential anti-inflammatory and antioxidant drugs for clinical frontiers and promotes the research progress of curcumin analogues.

The chlorinated monocarbonyl curcumin compounds CAK01-CAK07 are designed and synthesized by using aldehydes and ketones as raw materials, and the more stable monocarbonyl curcumin analogues are obtained by replacing the unstable β-dicarbonyl structure with monocarbonyl, and the liposoluble groups are introduced into the structure and chlorine substitution is carried out to make it have better pharmacokinetic behavior and higher anti-inflammatory, antioxidant, and antidepressant activities.

The chlorinated monocarbonyl curcumin compounds CAK01-CAK07 in the invention have the advantages of novel structure, simple synthesis method, suitable for large-scale production, strong anti-inflammatory and antioxidant activity.

### BRIEF DESCRIPTION OF THE DRAWINGS

FIGS. 1A-1H are the determinations of ABTS free radical capture ability of CAK01-CAK07.

FIGS. 2A-2H show the inhibitory effects of CAK01-CAK07 on H₂O₂-induced oxidative damage in PC12 cells.

FIGS. 3A-3H show the inhibitory effects of CAK01-CAK07 on CORT-induced PC12 cell damage.

FIGS. 4A-4G show the inhibitory effects of CAK06 on LPS-induced BV2 cell stress.

FIGS. 5A-5B are improvement effects of CAK06 on depression-like behavior in CUMS depression model mice.

FIGS. 6A-6E are expression effects of CAK06 on oxidative stress and synaptic-related protein in the hippocampus of CUMS depression model mice.

FIG. 7 is a safety evaluation of CAK06 on HE staining of liver, heart, spleen, lung, and kidney tissues in mice.

### DETAILED DESCRIPTION OF THE EMBODIMENTS

The following is a further explanation of the invention in combination with drawings and examples.

### Example 1

Preparation of chlorinated monocarbonyl curcumin compounds CAK01-CAK07

6 parts of 3,5-dichloro-4-hydroxybenzaldehyde (also can be 2 or 10 parts) and 3 parts of 4-piperidone (also can be 1 or 5 parts) were dissolved in 32 parts (also can be 15 or 50 parts) of saturated hydrogen chloride glacial acetic acid solution, and stirred at 25° C. (can also be 22° C. or 28° C.) for 18 h (can also be 12 h or 24 h) to obtain solution A, after standing for 18 h (can also be 12 h or 24 h), the solution A was filtered to obtain the precipitate, and the precipitate was repeatedly washed with deionized water and anhydrous ethanol for 3 times (can also be 2 or 4 times), the remaining precipitate was dried to obtain compound CAK01, and its structural formula was:

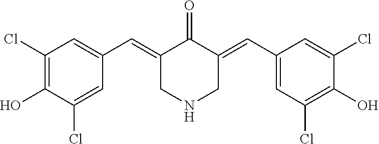 [SMILES: O=C1/C(=C/c2cc(Cl)c(O)c(Cl)c2)CNC/C1=C\c1cc(Cl)c(O)c(Cl)c1]

6 parts of 3,5-dichloro-4-hydroxybenzaldehyde (also can be 2 or 10 parts) and 3 parts of 1-methyl-4-piperidone (also can be 1 or 5 parts) were dissolved in 32 parts (also can be 15 or 50 parts) of saturated hydrogen chloride glacial acetic acid solution, and stirred at 25° C. (also can be 22° C. or 28° C.) for 18 h (also can be 12 h or 24 h) to obtain solution B, after standing for 18 h (also can be 12 h or 24 h), solution B was filtered to obtain precipitate, and the precipitate was repeatedly washed with deionized water and anhydrous ethanol for 3 times (also can be 2 or 4 times), the remaining precipitate is dried to obtain compound CAK02, and its structural formula is:

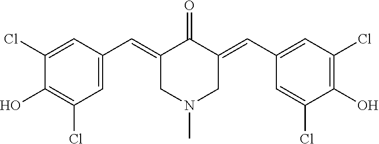 [SMILES: CN1C/C(=C\c2cc(Cl)c(O)c(Cl)c2)C(=O)/C(=C/c2cc(Cl)c(O)c(Cl)c2)C1]

6 parts of 3,5-dichloro-4-hydroxybenzaldehyde (also 2 parts or 10 parts) and 3 parts of 1-ethyl-4-piperidone (also 1 part or 5 parts) were dissolved in 32 parts (also 15 parts or 50 parts) of saturated hydrogen chloride glacial acetic acid solution, and stirred at 25° C. (also 22° C. or 28° C.) for 18 h (also 12 h or 24 h) to obtain solution C, after standing for 18 h (also 12 h or 24 h), solution C was filtered to precipitate, and the precipitate was washed three times (also 2 times or 4 times) with deionized water and anhydrous ethanol, the remaining precipitate is dried to obtain compound CAK03, and its structural formula is:

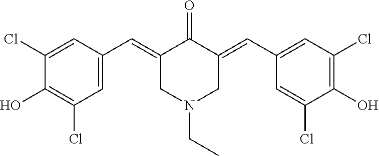 [SMILES: CCN1C/C(=C\c2cc(Cl)c(O)c(Cl)c2)C(=O)/C(=C/c2cc(Cl)c(O)c(Cl)c2)C1]

6 parts of 3,5-dichloro-4-hydroxybenzaldehyde (also 2 parts or 10 parts) and 3 parts of 1-isopropyl-4-piperidone (also 1 part or 5 parts) were dissolved in 32 parts (also 15 parts or 50 parts) of saturated hydrogen chloride glacial acetic acid solution, and stirred at 25° C. (also 22° C. or 28° C.) for 18 h (also 12 h or 24 h) to obtain solution D, after standing for 18 h (also 12 h or 24 h), solution D was filtered to precipitate, and the precipitate was washed three times (also 2 times or 4 times) with deionized water and anhydrous ethanol, the remaining precipitate is dried to obtain compound CAK04, and its structural formula is:

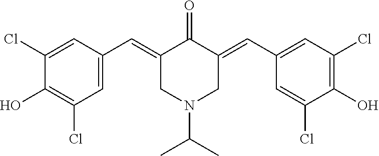 [SMILES: CC(C)N1C/C(=C\c2cc(Cl)c(O)c(Cl)c2)C(=O)/C(=C/c2cc(Cl)c(O)c(Cl)c2)C1]

6 parts of 3-chloro-4-hydroxy-5-methoxybenzaldehyde (also can be 2 or 10 parts) and 3 parts of 1-isopropyl-4-piperidone (also can be 1 or 5 parts) were dissolved in 32 parts (also can be 15 or 50 parts) of saturated hydrogen chloride glacial acetic acid solution, and stirred at 25° C. (also can be 22° C. or 28° C.) for 18 h (also can be 12 h or 24 h) to obtain solution E, after standing for 18 h (also can be 12 h or 24 h), solution E was filtered to obtain the precipitate, and the precipitate was repeatedly washed with deionized water and anhydrous ethanol for 3 times (also can be 2 or 4 times), the remaining precipitate is dried to obtain compound CAK05, and its structural formula is:

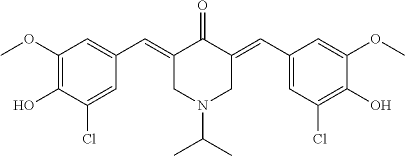

6 parts of 3-chloro-4-hydroxy-5-methoxybenzaldehyde (also can be 2 parts or 10 parts) and 3 parts of 4-piperidone (also can be 1 portion or 5 parts) were dissolved in 32 parts (also can be 15 parts or 50 parts) of saturated hydrogen chloride glacial acetic acid solution, and stirred at 25° C. (also can be 22° C. or 28° C.) for 18 h (also can be 12 h or 24 h) to obtain solution F, after standing for 18 h (also can be 12 h or 24 h), the solution F was filtered to obtain the precipitate, the precipitate was repeatedly washed with deionized water and anhydrous ethanol for 3 times (also can be 2 times or 4 times), and the remaining precipitate was dried to obtain compound CAK06, and its structural formula is:

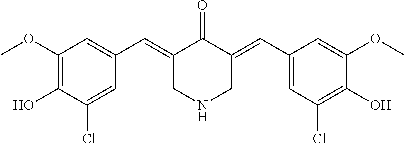

6 parts of 3-chloro-4-hydroxy-5-methoxybenzaldehyde (also can be 2 or 10 parts) and 3 parts of 1-methyl-4-piperidone (also can be 1 or 5 parts) were dissolved in 32 parts (also can be 15 or 50 parts) of saturated hydrogen chloride glacial acetic acid solution, and stirred at 25° C. (also can be 22° C. or 28° C.) for 18 h (also can be 12 h or 24 h) to obtain solution G, after standing for 18 h (also can be 12 h or 24 h), the solution G was filtered to obtain the precipitate, and the precipitate was repeatedly washed with deionized water and anhydrous ethanol for 3 times (also can be 2 or 4 times), the remaining precipitate is dried to obtain compound CAK07, and its structural formula is:

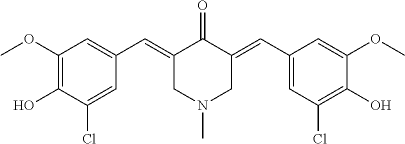

### Example 2

Characterization of chlorinated monocarbonyl curcumin compounds CAK01-CAK07

### 1. Experimental Materials

The chlorinated monocarbonyl curcumin compounds CAK01-CAK07 were prepared under the conditions in Example 1.

### 2. Experimental Methods

The chlorinated monocarbonyl curcumin compounds CAK01-CAK07 prepared under the conditions of Example 1 were taken for nuclear magnetic resonance, mass spectrometry, solubility, and melting point detection.

### 3. Experimental Results

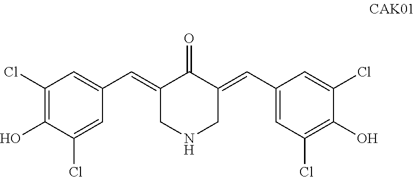 [SMILES: O=C1/C(=C/c2cc(Cl)c(O)c(Cl)c2)CNC/C1=C\c1cc(Cl)c(O)c(Cl)c1]

3,5-bis ((E)-3,5-dichloro-4-hydroxybenzylidene) piperidin-4-one

The yield was 49.6%, the melting range was 293-294° C., and the solubility was 0.17 mg/100 g,
structural characterization: ¹H NMR (400 MHZ, DMSO-d6) δ10.74 (s, 2H), 9.81 (s, 1H), 7.73 (s, 2H), 7.61 (s, 4H), 4.48 (s, 4H). HRMS calcd for C₁₉H₁₃Cl₄NO₃ [M+H]⁺443.9723, observed 443.9712.

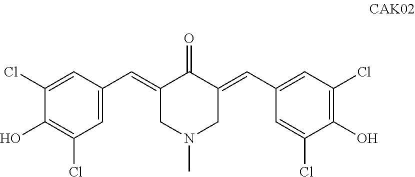 [SMILES: CN1C/C(=C\c2cc(Cl)c(O)c(Cl)c2)C(=O)/C(=C/c2cc(Cl)c(O)c(Cl)c2)C1]

3,5-bis ((E)-3,5-dichloro-4-hydroxybenzylidene)-1-methylpiperidin-4-one

The yield was 34.62%, the melting range was 280-281° C., and the solubility was 0.09 mg/100 g,
structural characterization: ¹H NMR (400 MHZ, DMSO-d6) δ 7.71 (s, 2H), 7.59 (s, 4H), 4.52 (s, 4H), 2.91 (s, 3H). HRMS calcd for C₂₀H₁₅Cl₄NO₃ [M+H]⁺457.9878, observed 457.9868.

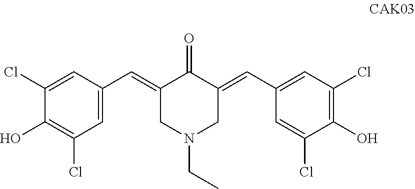 [SMILES: CCN1C/C(=C\c2cc(Cl)c(O)c(Cl)c2)C(=O)/C(=C/c2cc(Cl)c(O)c(Cl)c2)C1]

3,5-bis ((E)-3,5-dichloro-4-hydroxybenzylidene)-1-ethylpiperidin-4-one

The yield was 39.15%, the melting range was 292-293° C., and the solubility was 0.08 mg/100 g,
structural characterization: ¹H NMR (400 MHZ, DMSO-d6) δ 11.10 (s, 2H), 7.76 (s, 2H), 7.63 (s, 4H), 4.59 (s, 4H), 3.33 (s, 2H), 1.24 (s, 3H). HRMS calcd for C₂₁H₁₇Cl₄NO₃ [M+H]⁺472.0036, observed 472.0025.

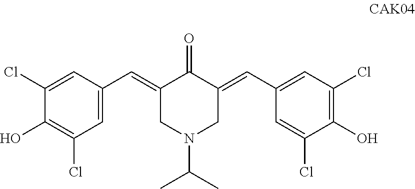 [SMILES: CC(C)N1C/C(=C\c2cc(Cl)c(O)c(Cl)c2)C(=O)/C(=C/c2cc(Cl)c(O)c(Cl)c2)C1]

3,5-bis ((E)-3,5-dichloro-4-hydroxybenzylidene)-1-isopropylpiperidin-4-one

The yield was 24.96%, the melting range was 290-291° C., and the solubility was 0.04 mg/100 g,

structural characterization: ¹H-NMR (400 MHZ, DMSO) δ 811.02 (s, 2H), 7.76 (s, 2H), 7.66 (s, 4H), 4.52 (s, 4H), 3.79 (s, 1H), 1.34 (s, 6H). HRMS calcd for C₂₂H₁₉Cl₄NO_{3 [}M+H]⁺487.1980, observed 487.0090.

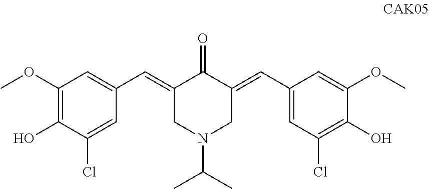

3,5-bis ((E)-3-chloro-4-hydroxy-5-methoxybenzylidene)-1-isopropylpiperidin-4-one

The yield was 49.37%, the melting range was 270-271° C., and the solubility was 0.14 mg/100 g,
structural characterization: ¹H-NMR (400 MHZ, DMSO) δ 10.23 (s, 2H), 7.81 (s, 2H), 7.26 (d, J=1.9 Hz, 2H), 7.17 (d, J=2.0 Hz, 2H), 3.92 (s, 6H), 3.44 (q, J=7.0 Hz, 4H), 1.35 (d, J=6.5 Hz, 6H). 1.06 (s, 1H). HRMS calcd for C₂₄H₂₅Cl₂NO₅ [M+H]⁺ 478.1183, observed 478.1178.

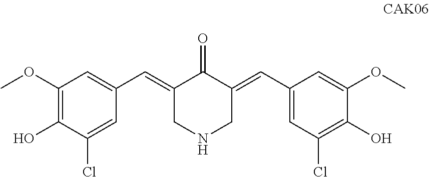

3,5-bis ((E)-3-chloro-4-hydroxy-5-methoxybenzylidene) piperidin-4-one

The yield was 56.33%, the melting range was 290-291° C., and the solubility was 171.77 mg/100 g,
structural characterization: ¹H-NMR (400 MHz, DMSO) δ 10.20 (s, 2H), 9.79 (s, 1H), 7.78 (d, J=2.1 Hz, 2H), 7.18 (s, 4H), 4.52 (s, 4H), 3.90 (s, 6H). HRMS calcd for C₂₁H₁₉Cl₂NO₅ [M+H]⁺436.0713, observed 436.0713.

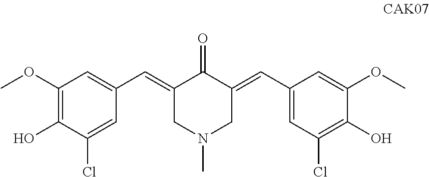

3,5-bis ((E)-3-chloro-4-hydroxy-5-methoxybenzylidene)-1-methylpiperidin-4-one

The yield was 51.29%, the melting range was 251-252° C., and the solubility was 34.77 mg/100 g,

structure characterization: ¹H-NMR (400 MHZ, DMSO) δ10.26 (s, 2H), 7.79 (s, 2H), 7.17 (d, J=12.7 Hz, 4H), 4.65 (s, 4H), 3.91 (s, 6H), 3.01 (s, 3H). HRMS calcd for C₂₂H₂₁Cl₂NO₅ [M+H]+ 450.0870, observed 450.0870.

### Example 3

Evaluation of the ability of chlorinated monocarbonyl curcumin compounds in capturing abts free radicals.

### 1. Experimental Materials

ABTS, K2S2O8 and PBS (Shanghai Aladdin Reagent Co., Ltd.), anhydrous ethanol, and ascorbic acid Vit C (Chengdu Manster Biotechnology Co., Ltd.) were used to prepare the chlorinated monocarbonyl curcumin compounds CAK01-CAK07 under the conditions of Example 1.

### 2. Experimental Methods

### Preparation of ABTS Working Solution

66.2 mg of K₂S₂O₈ was weighed and placed in a 100 mL volumetric flask, pure water was added to the scale line to prepare a 2.45 mmol/L K₂S₂O₈ solution. 192 mg of ABTS was accurately weighed and placed in a 25 mL volumetric flask, and the newly prepared K₂S₂O₈ solution was added for dissolving and constant volume dilution, after standing in the dark for 20 h, the ABTS mother liquor was obtained. Before use, to make the measured value more accurate, the ABTS mother liquor needs to be diluted with a PBS solution of pH7.4 until the absorbance range of the ABTS working solution at 734 nm is 0.70±0.02.

Drug concentration screening: To avoid the complete removal of free radicals by excessive drug concentration and the inability to compare the ability to capture ABTS free radicals, a preliminary screening of drug concentration is required before the formal test. A gradient concentration of compound solution was prepared, and a gradient concentration of Vit C solution was prepared as a positive control. A total of 450 μL of ABTS working solution was accurately absorbed into a 1.5 mL EP tube, and 150 μL of the gradient solution of each group of drugs was added, after 20 min of reaction in the dark, 150 μL of the reaction solution was taken and placed in a 96-well plate, the absorbance was measured by a microplate reader. Three duplicate wells were set for each concentration, and the drug concentration with absorbance between 0.2-0.7 was selected for subsequent determination.

Determination operation: After the drug concentration screening was completed, 7 concentration gradient groups were set within this concentration range. A total of 450 μL of ABTS working solution was accurately absorbed into a 1.5 mL EP tube, and 150 μL of the gradient solution of each group of drugs was added. After 20 min of reaction in the dark, 150 μL of the reaction solution was taken and placed in a 96-well plate, the absorbance was measured by a microplate reader. Three duplicate wells were set for each concentration, the positive control group was a gradient solution of Vit C, and the blank control group was an equal volume of anhydrous ethanol.

### 3. Experimental Results

TABLE 1
IC₅₀ values of ABTS captured by Vit C and CAK01-07

| Compound | Vit C | CAK01 | CAK02 | CAK03 | CAK04 | CAK05 | CAK06 | CAK07 |
|---|---|---|---|---|---|---|---|---|
| IC₅₀ | 54.54 | 79.62 | 223.7 | 46.56 | 57.78 | 369.1 | 44.95 | 197.3 |
| (μg/mL) | | | | | | | | |

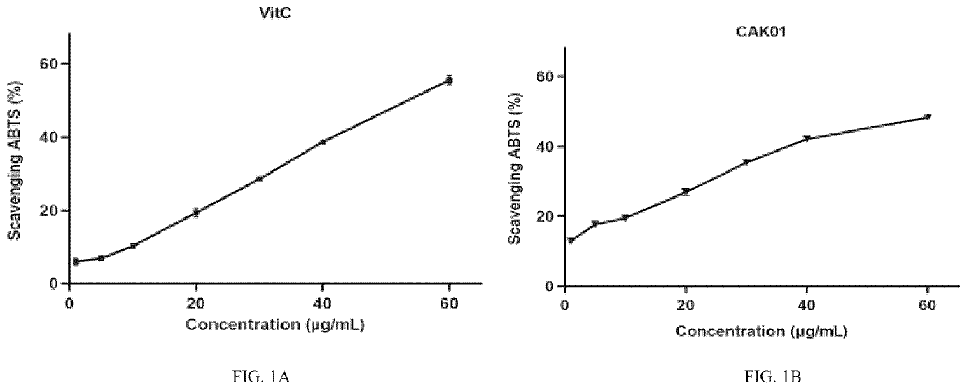

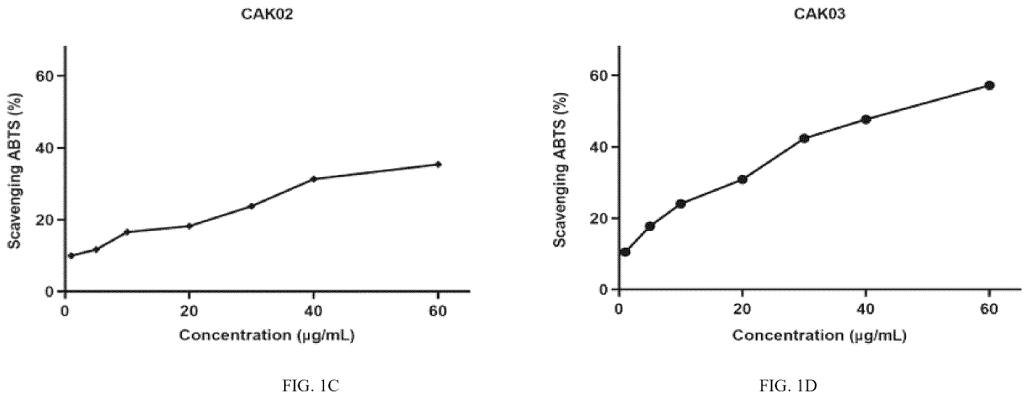

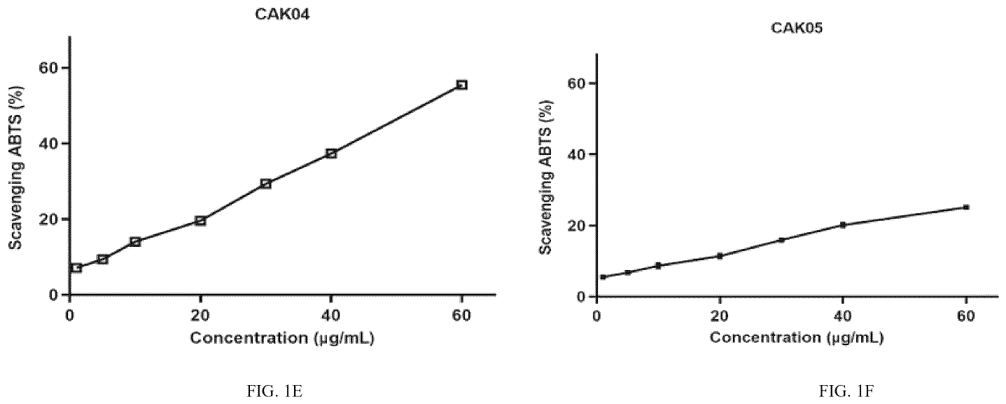

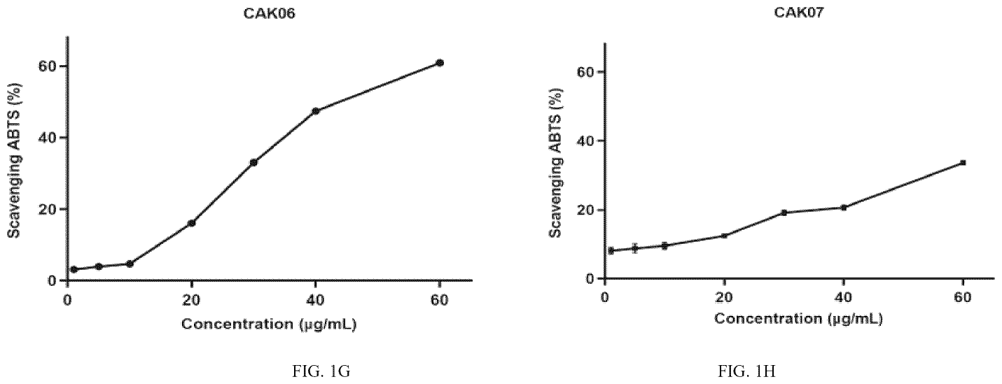

The experimental results are shown in Table 1 and FIGS. 1A-1H. As the drug concentration increases, the ability of each compound in capturing ABTS free radicals gradually increases. Compared with the IC₅₀ value of the strong antioxidant Vit C (54.54 μg/mL), CAK03 (46.56 μg/mL) and CAK06 (44.95 μg/mL) show stronger antioxidant capacity, the antioxidant capacity of CAK04 (57.78 μg/mL) is comparable to that of Vit C, and the remaining compounds also show a certain ability in capturing ABTS oxygen free radicals.

### Example 4

Safety evaluation of chloromonocarbonyl curcumin compounds on PC12 nerve cells

### 1. Experimental Materials

PC12 cells (Basic Medical Experimental Center, Affiliated Hospital of Southwest Medical University), the curcumin compounds CAK01-CAK07 in Example 1.

### 2. Experimental Methods

The toxicity of each drug to PC12 cells was determined by MTT assay. The principle of the MTT colorimetric method: there is succinate dehydrogenase in the mitochondria of living cells, which can make exogenous MTT become blue-purple crystal formazan insoluble in water, this phenomenon cannot be observed in dead cells. The blue-purple crystal formazan can be dissolved by DMSO, and the absorbance value is measured by a microplate reader at a wavelength of 490 nm, which in turn reflects the number of living cells. When the absorbance is lower, the corresponding living cells are less, indicating that the toxicity of the tested drug to the cells is stronger. The operation method is as follows:

The cells were resuscitated with DMEM high glucose medium containing 10% fetal bovine serum and cultured in a 5% CO₂ cell incubator at 37° C., when the cells grew to the logarithmic growth phase, they were inoculated into a 96-well plate and cultured for 24 hours, each well was added with 100 μL of each compound solution at the corresponding gradient concentration (60, 30, 15, 7.5, 3.75 μM), after incubation in the incubator for 24 h or 48 h, 20 μL of MTT was added to continue incubation for 4 h, the supernatant was discarded and 150 μL of DMSO was added to dissolve the blue-purple crystals, after shaking for 10 min, the cell viability was detected by a microplate reader, and classified according to the cytotoxicity standard. The relevant calculation formula is as follows:

$$Cell proliferation rate ( \% ) = [ \frac{{A}_{Experment group} - {A}_{Blank group}}{{A}_{Control group} - {A}_{Blank group}} ] \times 100 \%$$

### 3. Experimental Results

TABLE 2
Proliferation rate of curcumin analogues on PC12 cells
(%) (x̅ ± s, n = 3)

| | Drug | | | | |
|---|---|---|---|---|---|
| | concentration | Relative growth rate (%) | | | |
| Drug | (μM) | 24 h | Rank | 48 h | Rank |
| CAK01 | 3.75 | 104.22 ± 1.99 | 0 | 99.22 ± 1.65 | 1 |
| | 7.5 | 101.17 ± 2.97 | 0 | 100.73 ± 3.19 | 0 |
| | 15 | 99.46 ± 2.16 | 1 | 99.33 ± 1.79 | 1 |
| | 30 | 101.62 ± 3.23 | 0 | 100.73 ± 2.88 | 0 |
| | 60 | 104.04 ± 1.43 | 0 | 85.41 ± 3.45 | 1 |
| | 3.75 | 102.93 ± 1.13 | 0 | 98.35 ± 1.84 | 1 |
| | 7.5 | 99.40 ± 4.11 | 1 | 100.22 ± 3.73 | 0 |
| CAK02 | 15 | 95.08 ± 10.06 | 1 | 95.22 ± 1.86 | 1 |
| | 30 | 99.48 ± 1.91 | 1 | 97.59 ± 8.36 | 1 |
| | 60 | 88.18 ± 0.93 | 1 | 98.48 ± 2.00 | 1 |
| | 3.75 | 96.25 ± 0.78 | 1 | 98.96 ± 1.23 | 1 |
| | 7.5 | 87.65 ± 5.86 | 1 | 94.59 ± 1.02 | 1 |
| CAK03 | 15 | 88.25 ± 5.62 | 1 | 89.84 ± 0.82 | 1 |
| | 30 | 82.96 ± 2.83 | 1 | 95.25 ± 9.43 | 1 |
| | 60 | 77.51 ± 4.22 | 1 | 30.33 ± 6.54 | 3 |
| | 3.75 | 98.54 ± 5.42 | 1 | 100.97 ± 3.25 | 0 |
| | 7.5 | 99.95 ± 5.76 | 1 | 100.45 ± 2.96 | 0 |
| CAK04 | 15 | 99.06 ± 5.73 | 1 | 99.75 ± 1.57 | 1 |
| | 30 | 99.66 ± 2.34 | 1 | 98.02 ± 3.00 | 1 |
| | 60 | 101.64 ± 3.95 | 0 | 100.17 ± 2.92 | 0 |
| | 3.75 | 89.59 ± 2.85 | 1 | 61.38 ± 0.91 | 2 |
| | 7.5 | 68.98 ± 2.27 | 2 | 51.45 ± 1.44 | 2 |
| CAK05 | 15 | 62.32 ± 3.54 | 2 | 22.26 ± 1.14 | 3 |
| | 30 | 56.50 ± 1.99 | 2 | 15.43 ± 0.32 | 4 |
| | 60 | 41.84 ± 0.40 | 3 | 11.06 ± 0.87 | 4 |
| CAK06 | 3.75 | 98.50 ± 3.77 | 0 | 100.05 ± 0.75 | 0 |
| | 7.5 | 100.42 ± 2.85 | 0 | 92.97 ± 2.54 | 1 |
| | 15 | 98.55 ± 4.12 | 1 | 88.82 ± 0.28 | 1 |
| | 30 | 94.56 ± 2.82 | 1 | 93.60 ± 4.14 | 1 |
| | 60 | 114.78 ± 1.81 | 0 | 109.26 ± 4.07 | 0 |
| | 3.75 | 99.04 ± 1.89 | 1 | 82.45 ± 2.34 | 1 |
| | 7.5 | 76.49 ± 0.75 | 1 | 46.41 ± 0.69 | 3 |
| CAK07 | 15 | 60.78 ± 0.55 | 2 | 30.99 ± 0.02 | 3 |
| | 30 | 53.24 ± 0.51 | 2 | 30.46 ± 0.54 | 3 |
| | 60 | 49.53 ± 0.31 | 3 | 24.20 ± 0.74 | 4 |

PC12 cells are a kind of rat adrenal medulla pheochromocytoma cells, the results of the effects of each compound on PC12 are shown in Table 2. When the concentration of CAK03 is 60 μM, the relative growth rate of PC12 cells decreases to 30.33±6.54% after 48 h, and the activity of PC12 cells is good at other time points and concentrations, CAK05 had a great influence on the activity of PC12 cells, when CAK05 is 7.5μM, the cell viability decreases to 68.98±2.27% after 24 h, when CAK05 is 3.75 μM, the cell viability decreases to 61.38±0.91% after 48 h. The toxicity of CAK07 is also high, when the concentration is 15 μM, the cell viability decreases to 60.78±0.55% after 24 h, and when the concentration is 7.5 μM, the cell viability decreases to 46.41±0.69% after 48 h. Relatively speaking, CAK01, CAK02, CAK04, and CAK06 have no significant cytotoxicity to PC12 cells at each time point and concentration, and those components are safe. The above results suggest that CAK01, CAK02, CAK04, and CAK06 in the series of compounds have good safety to central nervous cells and are potential central nervous system drug candidates; CAK05 and CAK07 are more toxic to cells and may be candidate drugs for the treatment of glioma.

### Example 5

Evaluation of the inhibitory effects of chlorinated monocarbonyl curcumin compounds CAK01-CAK07 on H₂O₂-induced oxidative damage on PC12 cells.

### 1. Experimental Materials

PC12 cells (Basic Medical Experimental Center of the Affiliated Hospital of Southwest Medical University), H₂O₂ (Shanghai McLean Biochemical Technology Co., Ltd.), and the curcumin compounds CAK01-CAK07 in Example 1.

### 2. Experimental Methods

Establishment of the model: Cells exposed to high concentrations of H₂O₂ can cause cell damage, which corresponds to the current oxidative stress theory of depression. The specific operation of the establishment of the model is as follows: after the cells grew to the logarithmic growth phase, they were inoculated in a 96-well plate and adhered to the wall for 24 hours, the blank control group and the H2O2 series concentration group (500, 550, 600, 650, 700, 750 μM) were set up. 100 μL of the corresponding complete medium was added to each group, and each group was set up with 3 duplicate wells, after 24 hours of culture in the incubator, the relative proliferation rate of the cells was treated according to Section 2.1 and the relative proliferation rate of the cells was calculated to find the best modeling concentration.

Drug evaluation: Cells in the logarithmic growth phase were inoculated in a 96-well plate, after 24 hours of adherent growth, blank control group, H₂O₂ model group, CAK01 group, CAK02 group, CAK03 group, CAK04 group, CAK05 group, CAK06 group, CAK07 group and positive drug fluoxetine (Flu) control group were set up, the blank control group was added with 100 μL of blank complete medium, and the H₂O₂ model group was added with 100 μL of H₂O₂ culture medium with a concentration of 675 μM, the administration groups were added with a mixture of drugs at concentrations of 3.75, 7.5, 15,30,60 μM and 675 μM of H₂O₂. After 24 hours of incubation in the incubator, the relative proliferation rates of the cells were calculated according to the method under Section 2.1.

### 3. Experimental Results

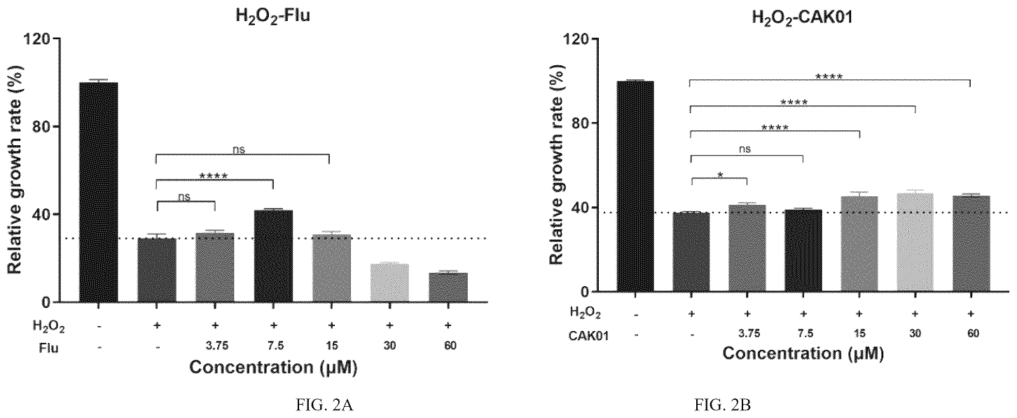

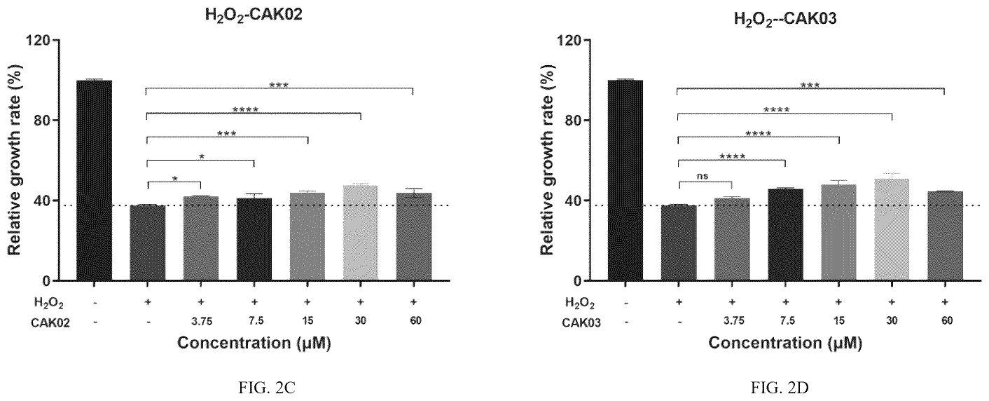

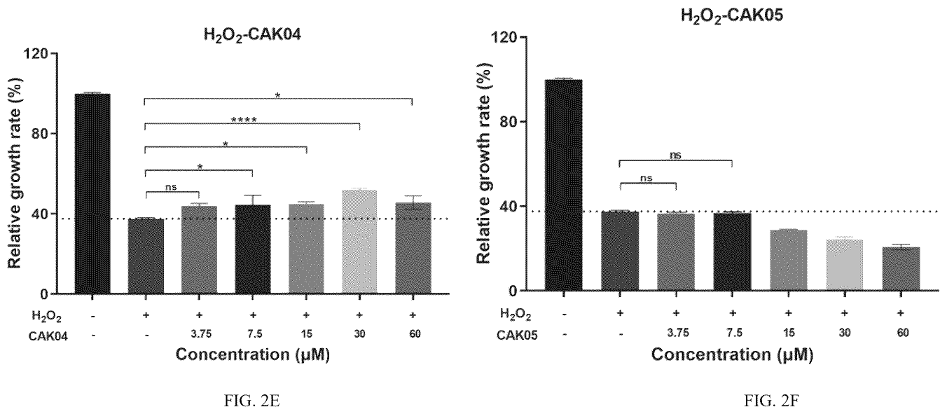

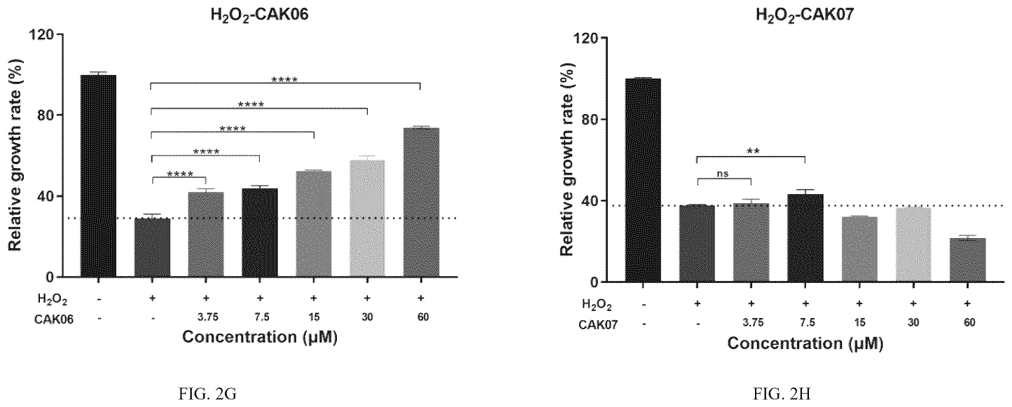

As a kind of exogenous stimulus, H₂O₂ can cause cells to produce a large amount of reactive oxygen species (ROS) when co-cultured with cells, and finally, cells appear apoptosis. The model was established by screening the appropriate H₂O₂ concentration, and then co-cultured with drugs to conduct a preliminary evaluation of drugs with potential antidepressant activity. PC12 cells were co-cultured with 3.75, 7.5, 15, 30, and 60 μM of drugs and 585.8 μM of H₂O₂ for 24 h, the positive drug group was Flu at the same concentration, the results of drug activity screening are shown in FIGS. 2A-2H. Compared with the H₂O₂ damage model group, Flu at a concentration of 7.5 μM significantly increased the cell survival rate to 41.92±0.66%; CAK01, CAK02, CAK03, and CAK04 increased the survival rate of cells in a concentration-dependent manner when the drug concentration was below 30 μM. Among them, CAK04 had the best effect, and the relative proliferation rate of cells was increased to 52.12±0.88% when the drug concentration was 30 μM, at higher concentrations, the cell survival rate may decrease due to the cytotoxicity of the drug itself. CAK05 failed to show the potential to restore cell viability in this model. CAK06 showed time-dependent and concentration-dependent cell protection after administration, and increased the cell proliferation rate to 73.91±0.69% at the highest concentration of 60 μM. CAK07 also has a certain cytoprotective effect at a drug concentration of 7.5 μM, but it may also inhibit cell growth at higher concentrations due to the toxicity of the drug itself. In summary, compared with Flu, CAK06 showed a more significant protective effect on PC12 cells damaged by oxidative stress.

### Example 6

Evaluation of the inhibitory effect of different concentrations of CAK01-CAK07 on corticosterone-induced PC12 cell damage.

### 1. Experimental Materials

PC12 cells (Basic Medical Experimental Center of the Affiliated Hospital of Southwest Medical University), corticosterone (CORT, purchased from Shanghai McLean Biochemical Technology Co., Ltd.), and the curcumin compounds CAK01-CAK07 in Example 1.

### 2. Experimental Methods

Establishment of the model: HPA axis dysfunction appeared in patients with depression, and CORT levels were significantly increased. A high concentration of CORT can induce the damage of PC12 cells, which is often used in the screening of antidepressant drugs. The specific operation of the model was as follows: after the cells grew to the logarithmic growth phase, they were inoculated in a 96-well plate and adhered to the wall for 24 h, the blank control group and the CORT series concentration groups (350, 400, 450, 500, 550, 600 μM) were set up, each group was added with 100 μL of the corresponding complete medium, each group was set up with three duplicate wells, after 24 h of culture in the incubator, the relative proliferation rate of the cells was treated and calculated according to the method in Section 2.1 to find out the best modeling concentration.

Drug evaluation: Cells in the logarithmic growth phase were inoculated in the 96-well plate, after 24 hours of adherent growth, blank control group, CORT model group, CAK01 group, CAK02 group, CAK03 group, CAK04 group, CAK05 group, CAK06 group, CAK07 group and positive drug Flu control group were set up, the blank control group was added with 100 μL of blank complete medium, and the CORT model group was added with 100 μL H₂O₂ of culture medium with a concentration of 500 μM, the administration group was added with a mixture of drugs at concentrations of 3.75, 7.5, 15, 30, 60 μM and 500 μM H₂O₂, after 24 hours of incubation in the incubator, the relative proliferation rate of the cells was calculated according to the method under Section 2.1.

### 3. Experimental Results

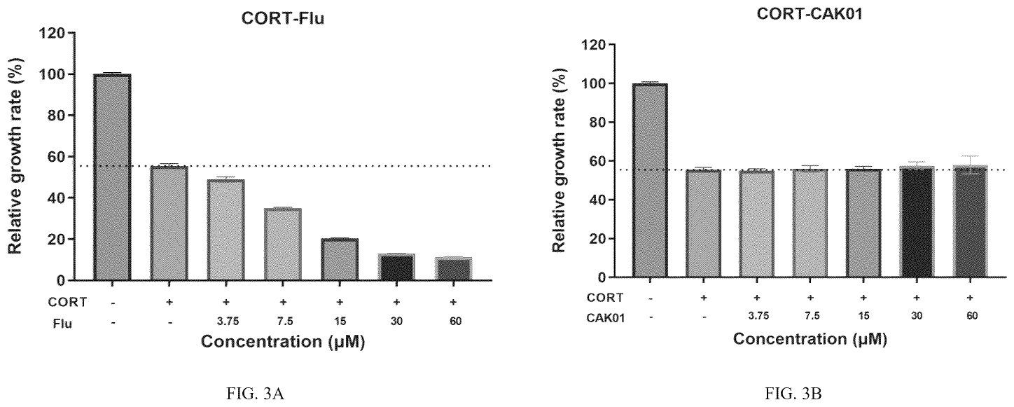

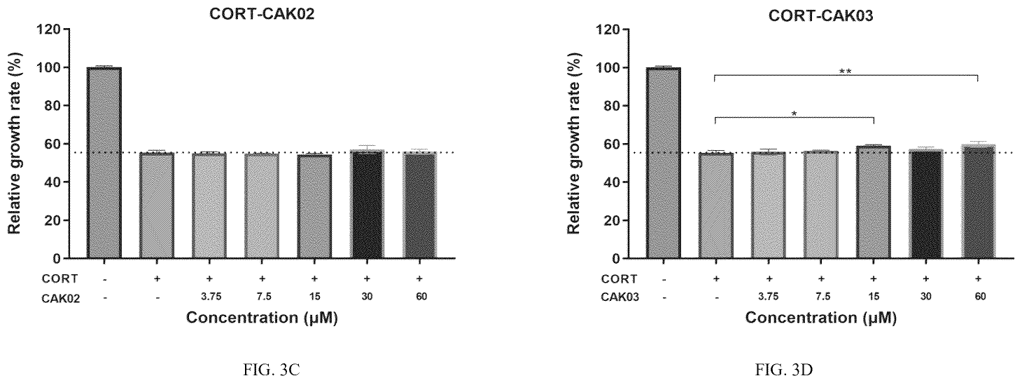

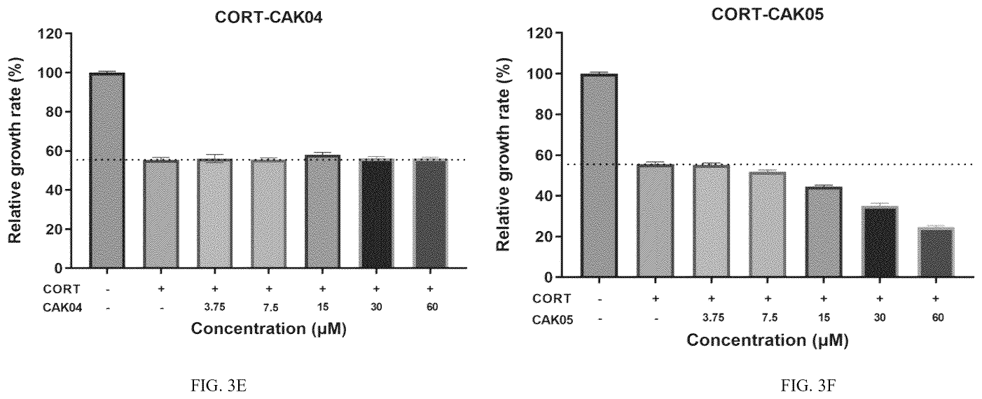

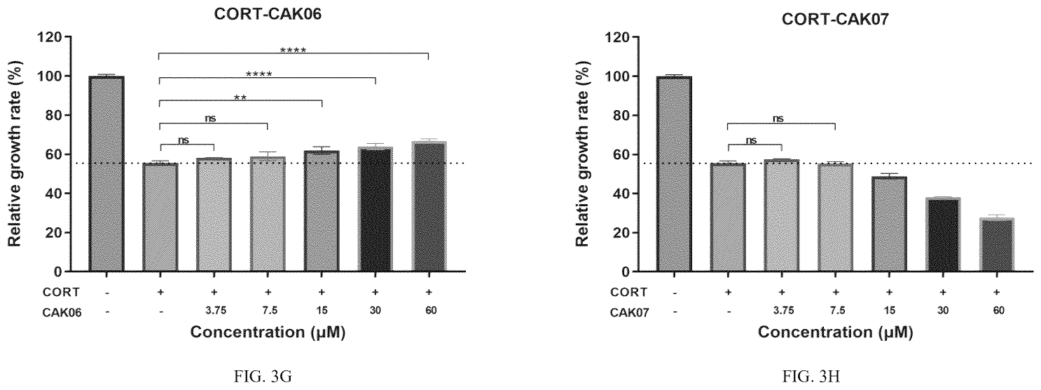

PC12 cells were co-cultured with 3.75, 7.5, 15, 30, 60 μM drugs and 514.6 μM CORT for 24 h, the positive drug group was Flu at the same concentration, the results of drug activity screening are shown in FIGS. 3A-3H. The CORT damage model group reduced the relative proliferation rate of cells to 55.48±1.16%, the Flu group did not show the effect of restoring CORT-damaged cells in the concentration range, but further inhibited cell growth due to its inherent drug toxicity. Similarly, CAK01, CAK02, CAK04, CAK05, and CAK07 did not show the potential to restore cell viability in this model. When the drug concentration was 15 μM and 60 μM, CAK03 significantly increased the relative cell proliferation rate to 59.15±0.51% and 59.92±1.53%, respectively. CAK06 showed time-dependent and concentration-dependent cell protection after administration, and the cell proliferation rate was significantly increased to 66.95±0.97% at the highest concentration of 60 μM. In general, CAK06 showed the most significant protective effect on CORT-damaged PC12 cells.

### Example 7

The inhibitory effect of CAK06 on LPS-induced BV2 cell stress

### 1. Experimental Materials

NO detection kit (Biyun Tian Biological Co., Ltd.), ROS detection kit (Biyun Tian Biological Co., Ltd.), lipopolysaccharide (LPS, purchased from Shanghai McLin Biochemical Technology Co., Ltd.), BV2 cells (Basic Medical Experimental Center of the Affiliated Hospital of Southwest Medical University), and the curcumin analogue CAK06 preferred in Example 6.

### 2. Experimental Methods

When the body is in a long-term stress and stress state, a large number of oxidizing substances and inflammatory factors accumulate in the body, causing depression and inducing depression-like symptoms. LPS is a pro-inflammatory substance, which can induce the increase of inflammatory factors, the accumulation of reactive oxygen species ROS and the release of NO in BV2 cells. The anti-inflammatory and antioxidant effects of drugs can be evaluated by establishing an LPS stress model.

NO detection: When BV2 cells grew to the logarithmic growth phase, they were inoculated in a 6-well plate at 1×10⁶ cells/well and cultured for 24 h. Then the original medium was replaced with CAK06 fresh DMEM high glucose medium containing high (10 μM), medium (5 μM), and low (1 μM) concentrations, and three duplicate wells were set up in each group. After the cells were treated with compound CAK06 for 12 h, the supernatant of the culture medium was centrifuged and stained according to the instructions of the NO detection kit, and the absorbance was detected at a wavelength of 540 nm.

ROS detection: The fluorescent dye probe DCFH-DA can freely pass through the BV2 cell membrane, and the dye entering the cell is enzymatically hydrolyzed to DCFH, at this time, it can no longer pass through the cell membrane. The intracellular ROS can oxidize DCFH to DCF with fluorescence, which can reflect the ROS content by detecting the fluorescence intensity of DCF. The specific operation is as follows: when BV2 cells grew to the logarithmic growth phase, they were inoculated in a 6-well plate at 1×10⁶ cells/well and cultured for 24 h. The blank control group, LPS model group, CAK06 low dose group (1 μM), CAK06 medium dose group (5 μM), and CAK06 high dose group (10 μM) were set up, each administration group also contained the corresponding concentration of LPS in the model group, and each group was set up three duplicate wells. After the cells were co-cultured with compound CAK06 for 12 h, the cells were stained according to the instructions of the ROS detection kit, and photographed and analyzed by an inverted fluorescence microscope.

### 3. Experimental Results

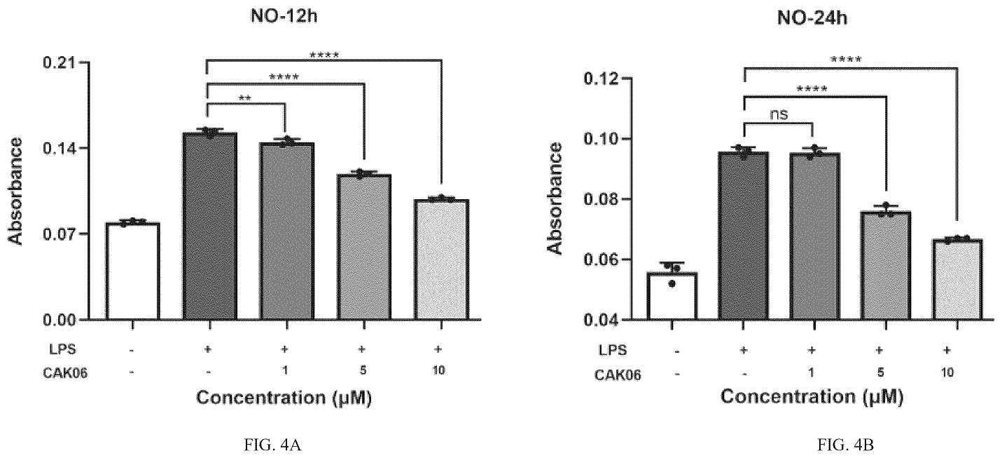

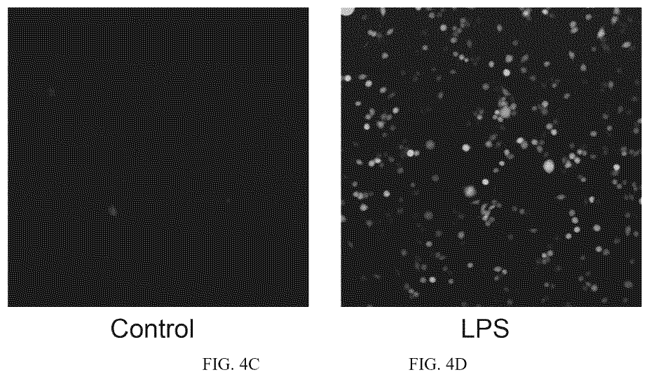

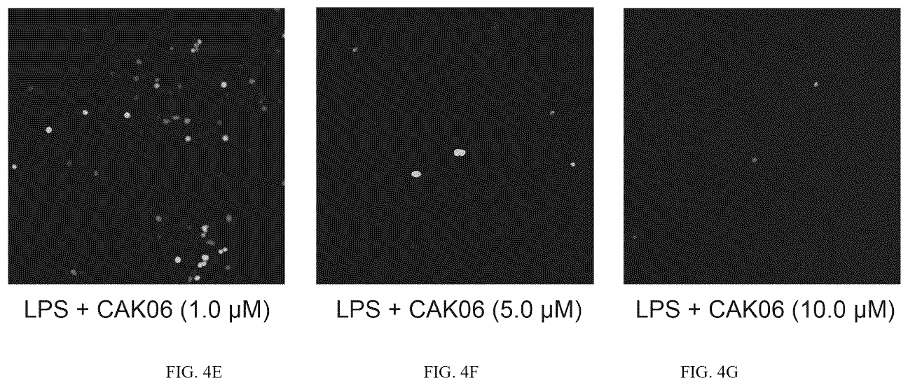

BV2 cells were co-cultured with 10, 5, and 1 μM of CAK06 and 1 μg/mL LPS for 12 h and 24 h, respectively, the results are shown in FIGS. 4A-4G. In the LPS model group, BV2 microglial cells were observed to release a large amount of NO due to external stimulation, and the absorbance at 12 h increased rapidly from 0.080 measured in the blank group to 0.153. After intervention with high, medium, and low doses of CAK06, the absorbance decreased to 0.099,0.119 and 0.145, respectively, indicating that CAK06 had the effect of reducing the toxic substance NO to protect cells. At 24 h, probably due to the consumption of LPS and drugs, the modeling effect of the model group was not as good as 12 h, and there was no significant difference between the low dose group and the model group, but the medium dose group and the high dose group were significantly improved. In the determination of reactive oxygen species ROS, it can be seen that after LPS intervention, the fluorescence intensity of the model group increased significantly under the color development of the fluorescent agent. After intervention with high, medium, and low doses of CAK06, the fluorescence intensity decreased in a concentration-dependent manner. CAK06 can be used to treat depression, anxiety, neuropathic pain, Alzheimer's disease, Parkinson's syndrome, cardiovascular disease, atherosclerosis, and other inflammation-related diseases for its powerful anti-inflammatory and antioxidant activity.

### Example 8

The effect of CAK06 on depression-like behavior in CUMS depression model mice.

### 1. Experimental Materials

C57BL/6J mice (body weight 18 g±2 g, male, purchased from Chongqing Tengxin Technology Laboratory Animal Co., Ltd.), Flu (Shanghai Macklin Biochemical Technology Co., Ltd.), the preferred curcumin analogue CAK06 in Example 6.

### 2. Experimental Methods

After one week of adaptive feeding, the newly purchased mice were divided into a blank group and CUMS model group. The mice in the blank group were not subjected to behavioral intervention and were fed normally. Mice in the model group were given 1-2 unpredictable behavioral stimuli every day. Stimulation methods are: fasting for 24 h; prohibition of water for 24 h; noise stimulation for 2 h; the cage box of mice was shaken violently for 10 min; forced swimming in ice water for 5 min, forced swimming in hot water for 5 min, and hanging upside down for 8 min, imprisoning in a 50 mL centrifuge tube for 4 h, electric shock for 2 min, continuous light for 48 h, and continuous dark for 48 h. When the same stimulation is continuous, the stimulation method is changed to ensure that the same stimulation is not continuous, and the model construction period is 42 days.

After the successful establishment of the mouse model was determined by behavioral evaluation, the depression-like mice were divided into a blank control group, a CUMS group, a Flu group, a CAK06 low dose group, and a CAK06 high dose group, with 5 mice in each group. Flu group mice were given 10 mg/kg Flu API by oral gavage, CAK06 low dose group mice were given 10 mg/kg CAK06 API, CAK06 high dose group mice were given 20 mg/kg CAK06 API, blank control group and CUMS group mice were given the same volume of pure water. The mice were administered once a day for 28 days, the behavioral evaluation of the mice was performed on the 14th day and the 28th day of administration, and the relevant indicators were measured after the end of the administration period.

### 3. Experimental Results

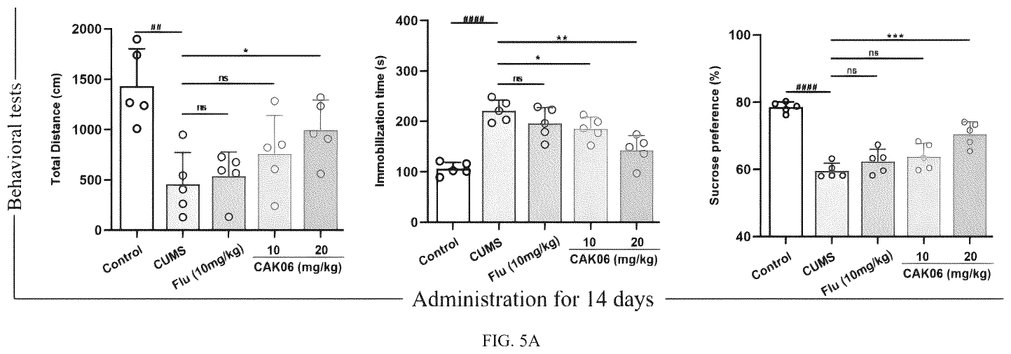

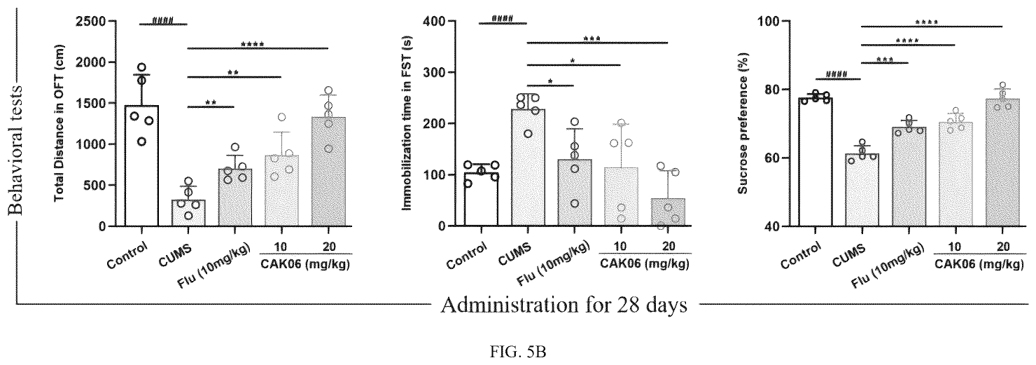

After the CUMS model was successfully established, CAK06 and fluoxetine were orally administered, on the 14th day, the mice were subjected to forced swimming and a sucrose preference test, the results are shown in FIG. 5A. In the forced swimming test, the Flu group did not show significant improvement in depressive symptoms, while the CAK06 group with a concentration of 10 mg/kg had a significant reduction in immobility time; meanwhile, with the increase of CAK06 dose, the CAK06 group with a concentration of 20 mg/kg showed stronger antidepressant effect. In the determination of sucrose preference, the Flu group and the low dose CAK06 group failed to significantly increase the sucrose preference rate, while the CAK06 group with a concentration of 20 mg/kg had a significant increase. It indicates that CAK06 has a dose-dependent antidepressant effect, and the onset speed is fast. The CAK06 group with a concentration of 20 mg/kg showed a significant antidepressant effect on the 14th day.

After 28 days of administration, the behavior of the mice was measured again, and the results are shown in FIG. 5B. Compared with the model group, the CAK06 group with a concentration of 10 mg/kg significantly increased the overall activity distance of depressed mice in the open field test (868.90±280.97 cm vs327.22±161.39 cm, P<0.01). The improvement of this behavior showed a good correlation with the dose of CAK06. With the increase of the dose, the effect of the CAK06 group with a concentration of 20 mg/kg was stronger (1336.66±264.87 cm vs327.22±161.39 cm, P<0.0001). Meanwhile, the mice in the Flu group also had considerable behavioral improvement, and the total activity distance of the mice increased to 705.43±159.14 cm, which was comparable to that of the low dose CAK06 group. The evaluation results of the forced swimming test showed that compared with the CUMS model group, the struggling movement desire of the mice in each administration group increased significantly. The immobility time of the mice in the Flu group decreased to 130.42±59.96 s, the immobility time of the mice in the low dose CAK06 group decreased to 114.86±84.07 s, and the immobility time of the mice in the high dose CAK06 group decreased to 54.44±53.38 s. Similarly, in the sucrose preference test, each administration group significantly improved the phenomenon of low sucrose water intake in depression-like mice, the sucrose preference rate of the model group was 61.36±2.18%, the sucrose preference rate of the fluoxetine group was 69.08±1.86%, the sucrose preference rate of the low dose CAK06 group was 70.62±2.33%, and the sucrose preference rate of the high dose CAK06 group was 77.42±2.77%.

Comprehensive comparison of the data of each group showed that CAK06 had a faster onset time and produced a rapid antidepressant effect in 14 days. After 28 days of the administration, the depression-like symptoms of mice in each administration group were improved, the low dose CAK06 group and the Flu group showed similar antidepressant effects, and the high dose CAK06 group had the best antidepressant effect. Flu at 10 mg/kg is the maximum clinically recommended dose, and higher doses may cause organ damage, while our drug CAK06 shows far more safety than Flu at the cellular level. Meanwhile, compared with our two CAK06 drug dose groups, the behavioral improvement of Flu was only comparable to that of the 10 mg/kg CAK06 dose group, which indicated that CAK06 had a better antidepressant effect than Flu, and the onset time was significantly shortened.

### Example 9

Effect of CAK06 on oxidative stress and synaptic-related protein expression in CUMS depression model mice

### 1. Experimental Materials

ACTIN antibody (ZENBIO), BDNF antibody, TrkB antibody, Nrf2 antibody, HO-1 antibody (BIOSS), the preferred curcumin analogue CAK06 in Example 6.

### 2. Experimental Methods

All experimental groups were taken 50 μL, 5×sodium dodecyl sulfate buffer (5×Loding buffer) was added according to the mass ratio of 4:1, the mixtures were fully mixed and placed in a 95° C. thermal cycler for 15 min, the denatured protein was stored at −80° C. for later use. The SDS-PAGE electrophoresis gel with a concentration of 10% was prepared, after the gel was fully solidified, the broken gel in the well was rinsed and loaded according to the protein concentration. At the beginning of electrophoresis, the voltage was set to 100 V and the electrophoresis lasted for 15 min, when the dye entered the separation gel, the voltage was changed to 180 V, and the electrophoresis continued until the dye reached the bottom of the gel. Then, the PVDF membrane was cut out according to the size of the separation gel, and the gel of the target protein was applied to a PVDF membrane, and then placed together in the membrane transfer solution for 10 min. After the power supply was turned on, the target proteins on the gel were all transferred to the PVDF membrane in an ice bath environment at a constant current of 200 mA for 1-2 h. The cleaned PVDF membrane was placed in an incubating box of 5% skimmed milk powder diluted by TBST Buffer and sealed on a shaker for 2 h (room temperature). After the closure was completed, the samples were washed three times (3×5 min) with TBST. Subsequently, the primary and secondary antibodies of each protein were incubated. Finally, the film was placed on the exposure plate and the luminescent liquid was added to the exposure imaging to quantify the relative expression of each protein.

### 3. Experimental Results

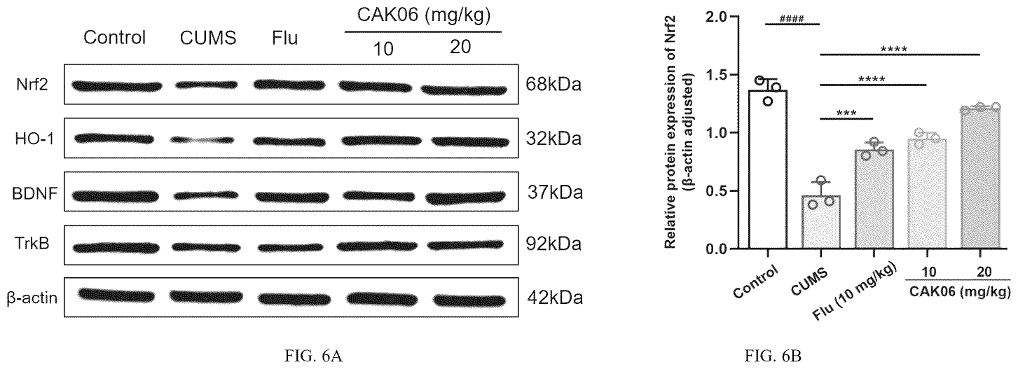

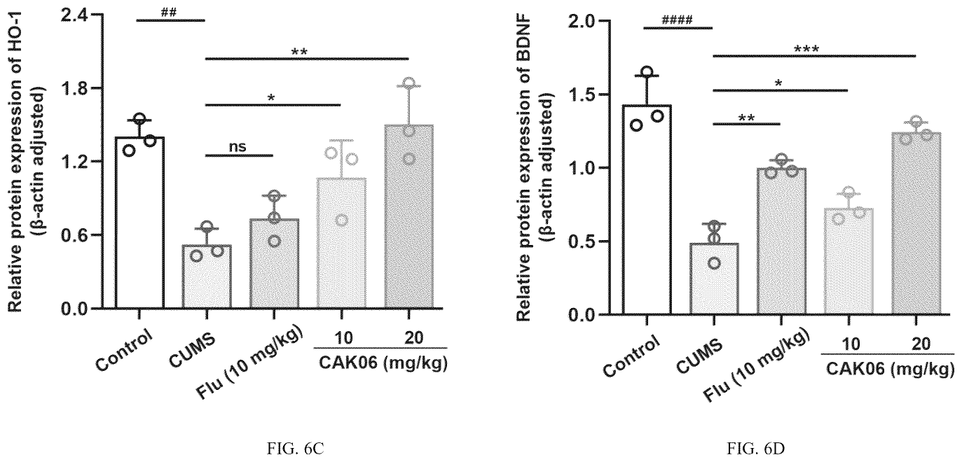

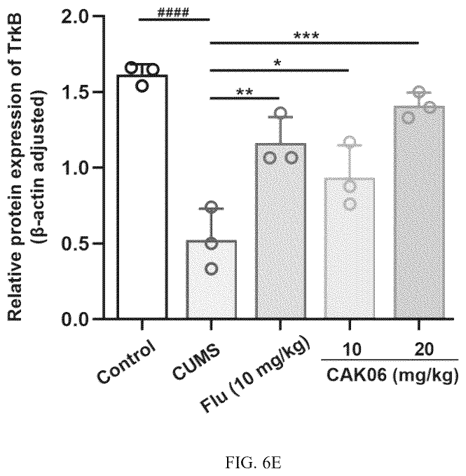

We measured the antioxidant and synaptic plasticity-related proteins Nrf2, HO-1, BDNF, and TrkB in the hippocampus, the results of western blot detection are shown in FIGS. 6A-6E. Nrf2 and HO-1 are key proteins in the classical antioxidant pathway. CAK06 may have a strong antioxidant and anti-inflammatory effect by affecting the Nrf2-HO-1 axis, which in turn promotes the expression of downstream synaptic plasticity-related proteins, restores synaptic signal transduction, and finally produces antidepressant efficacy. It is worth noting that the expression of these proteins in the Flu group also increased, especially the plasticity-related proteins. The expression of proteins in the Flu group was comparable to that in the low dose CAK06 group, and even better than that in the low dose group. Since the recovery of synaptic plasticity is closely related to the improvement of depressive symptoms, the improvement of depressive symptoms may also increase with the increase of plasticity protein expression, which is similar to the results observed in behavioral experiments, that is, the antidepressant effect of the low dose CAK06 group is close to that of Flu group. In the regulation of antioxidant-related proteins Nrf2 and HO-1, the CAK06 group shows better expression than the Flu group at low doses, which further indicates that the antioxidant effect of CAK06 is better than that of Flu, and the antioxidant effect may be the key to the antidepressant effect of CAK06.

### Example 10

HE staining safety evaluation results of CAK06 on liver, heart, spleen, lung, and kidney tissues of mice

### 1. Experimental Materials

4% paraformaldehyde fixative (Biosharp), hematoxylin dye (Wuhan Sevier Biotechnology Co., Ltd.), eosin dye (Wuhan Sevier Biotechnology Co., Ltd.), and the curcumin analogue CAK06 selected in Example 6.

### 2. Experimental Methods

After the mice were sacrificed, the hippocampus was taken out on ice and fixed with 4% paraformaldehyde fixative. Tissue dehydration, paraffin embedding, and tissue sections were tested. After dewaxing to water, the sections were stained with hematoxylin for 3 min, washed with water, differentiated with 1% hydrochloric acid alcohol, washed with water, and returned to blue with 1% ammonia water solution, washed with water. Subsequently, the sections were placed in 85% alcohol for 5 min, 95% alcohol for 5 min, anhydrous ethanol for 5 min, and eosin staining for 2 min. Finally, the dehydration sealing was carried out, and the digital slice scanner was used for image acquisition.

### 3. Experimental Results

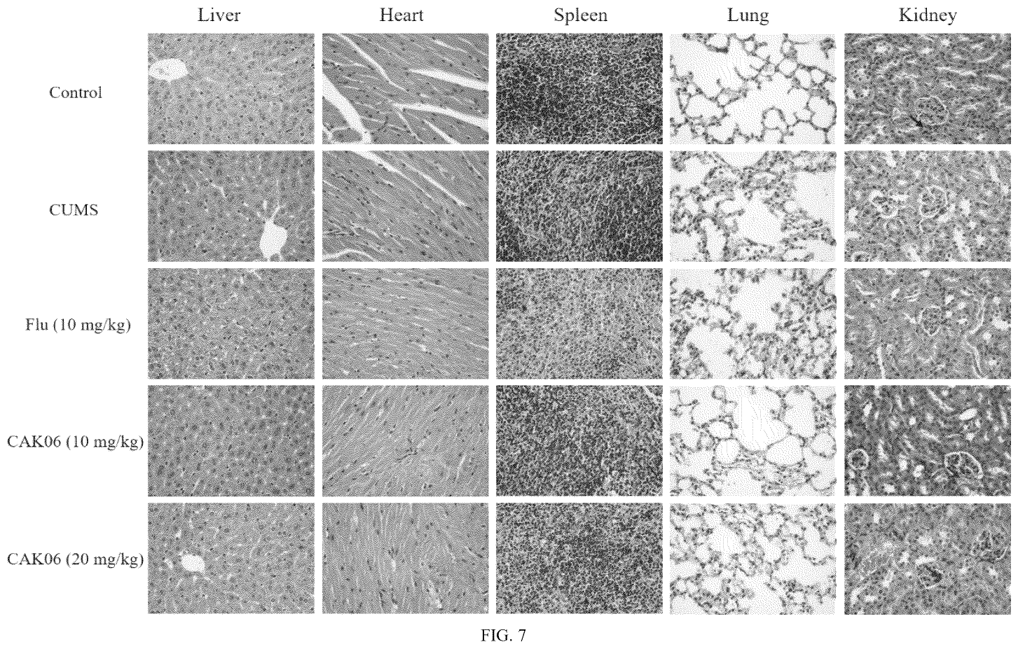

Through HE staining after tissue sections, we conducted a preliminary analysis of the pathological conditions of each tissue, the pathological staining is shown in FIG. 7. After staining, the nucleus in the tissue was blue, the cytoplasm was red, and the samples in each group were basically normal. Overall, this result proves that our drug has good safety.

### Example 11

Preparation of CAK06 oral solid dispersions

### 1. Experimental Materials

SD mice (220±20 g, purchased from the Experimental Animal Center of Southwest Medical University), PVP-k30 (Shanghai Aladdin Biochemical Technology Co., Ltd.), mannitol (Shanghai Aladdin Biochemical Technology Co., Ltd.), and the preferred curcumin analogue CAK06 in Example 6.

### 2. Experimental Method

15 mg of CAK06, 75 mg of PVP-k30, and 50 mg of mannitol were accurately weighed, the drug and excipients were placed in a 50 mL volumetric flask, and 20 mL of ethanol was added for ultrasonic dissolution, then, under the action of a constant temperature magnetic stirrer, the mixture was stirred at a speed of 500 rpm and a temperature of 50° C. for 30 min. After the reaction was sufficient, the solvent was evaporated by a rotary evaporator and dried overnight in an oven. After the solvent was completely evaporated, the drug was scraped off the wall of the volumetric flask, and the powder after grinding and sieving was sealed and stored.

### 3. Experimental Results

The optimal formulation of CAK06 oral solid dispersion was CAK06: 14.583 mg; pVP-k30: 129.481 mg; mannitol: 64.313 mg. The solubility of the optimal formulation of CAK06 oral solid dispersion was 16.82 mg/mL, which was nearly 10 times higher than that of CAK06 crude drug, it can be dissolved completely in 2 min. It can be used to treat inflammation-related diseases such as depression, Alzheimer's disease, neuropathic pain, and cardiovascular disease.

What is claimed is:

1. A chlorinated monocarbonyl curcumin compound, wherein the chlorinated monocarbonyl curcumin compound is represented by the following structural formula:

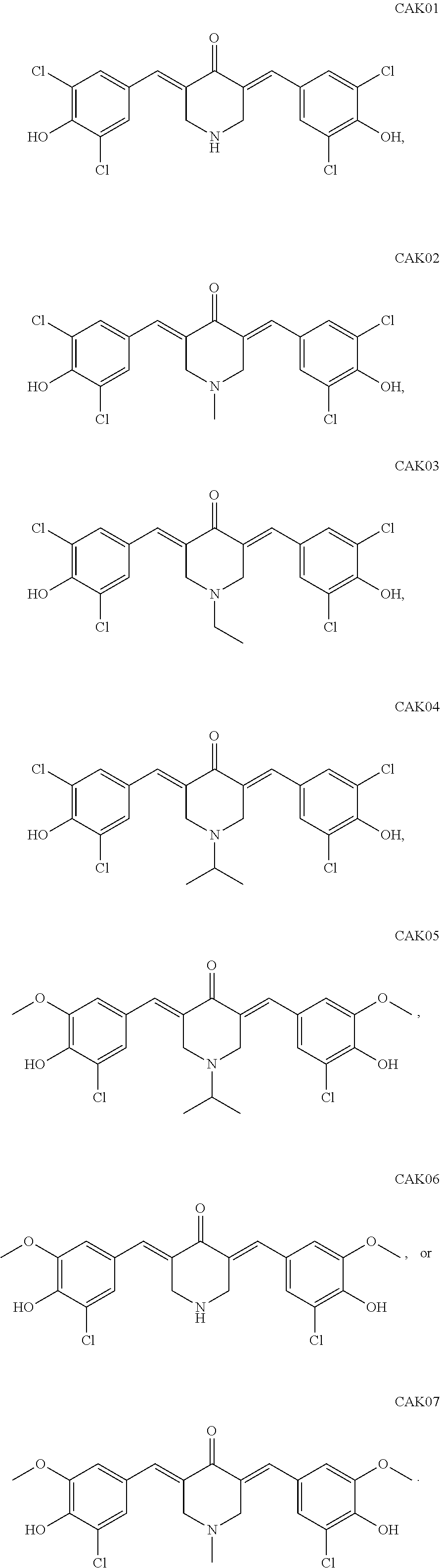

2. A preparation method for the chlorinated monocarbonyl curcumin compound according to claim 1, comprising:
dissolving 2-10 parts of 3,5-dichloro-4-hydroxybenzaldehyde and 1-5 parts of 4-piperidone in 15-50 parts of a saturated hydrogen chloride glacial acetic acid solution, stirring at 22-28° C. for 12-24 h to obtain a first solution, after standing for 12-24 h, filtering the first solution to obtain a first precipitate, washing the first precipitate repeatedly with deionized water and anhydrous ethanol for 2-4 times to obtain a first remaining precipitate, and drying the first remaining precipitate to obtain compound CAK01;
dissolving 2-10 parts of the 3,5-dichloro-4-hydroxybenzaldehyde and 1-5 parts of 1-methyl-4-piperidone in 15-50 parts of the saturated hydrogen chloride glacial acetic acid solution, stirring at 22-28° C. for 12-24 h to obtain a second solution, after standing for 12-24 h, filtering the second solution to obtain a second precipitate, washing the second precipitate repeatedly with the deionized water and the anhydrous ethanol for 2-4 times to obtain a second remaining precipitate, and drying the second remaining precipitate to obtain compound CAK02;
dissolving 2-10 parts of the 3,5-dichloro-4-hydroxybenzaldehyde and 1-5 parts of 1-ethyl-4-piperidone in 15-50 parts of the saturated hydrogen chloride glacial acetic acid solution, stirring at 22-28° C. for 12-24 h to obtain a third solution, after standing for 12-24 h, filtering the third solution to obtain a third precipitate, washing the third precipitate repeatedly with the deionized water and the anhydrous ethanol for 2-4 times to obtain a third remaining precipitate, and drying the third remaining precipitate to obtain compound CAK03;
dissolving 2-10 parts of the 3,5-dichloro-4-hydroxybenzaldehyde and 1-5 parts of 1-isopropyl-4-piperidone in 15-50 parts of the saturated hydrogen chloride glacial acetic acid solution, stirring at 22-28° C. for 12-24 h to obtain a fourth solution, after standing for 12-24 h, filtering the fourth solution to obtain a fourth precipitate, washing the fourth precipitate repeatedly with the deionized water and the anhydrous ethanol for 2-4 times to obtain a fourth remaining precipitate, and drying the fourth remaining precipitate to obtain compound CAK04;
dissolving 2-10 parts of 3-chloro-4-hydroxy-5-methoxybenzaldehyde and 1-5 parts of the 1-isopropyl-4-piperidone in 15-50 parts of the saturated hydrogen chloride glacial acetic acid solution, stirring at 22-28° C. for 12-24 h to obtain a fifth solution, after standing for 12-24 h, filtering the fifth solution to obtain a fifth precipitate, washing the fifth precipitate repeatedly with the deionized water and the anhydrous ethanol for 2-4 times to obtain a fifth remaining precipitate, and drying the fifth remaining precipitate to obtain compound CAK05;
dissolving 2-10 parts of the 3-chloro-4-hydroxy-5-methoxybenzaldehyde and 1-5 parts of the 4-piperidone in 15-50 parts of the saturated hydrogen chloride glacial acetic acid solution, stirring at 22-28° C. for 12-24 h to obtain a sixth solution, after standing for 12-24 h, filtering the sixth solution to obtain a sixth precipitate, washing the sixth precipitate repeatedly with the deionized water and the anhydrous ethanol for 2-4 times to obtain a sixth remaining precipitate, and drying the sixth remaining precipitate to obtain compound CAK06; and
dissolving 2-10 parts of the 3-chloro-4-hydroxy-5-methoxybenzaldehyde and 1-5 parts of the 1-methyl-4-piperidone in 15-50 parts of the saturated hydrogen chloride glacial acetic acid solution, stirring at 22-28° C. for 12-24 h to obtain a seventh solution, after standing for 12-24 h, filtering the seventh solution to obtain a seventh precipitate, washing the seventh precipitate repeatedly with the deionized water and the anhydrous ethanol for 2-4 times to obtain a seventh remaining precipitate, and drying the seventh remaining precipitate to obtain compound CAK07.

3. A method for preparing drugs for a treatment of inflammation-related diseases, comprising using the chlorinated monocarbonyl curcumin compound according to claim 1.

4. The method according to claim 3, wherein the inflammation-related diseases comprise depression, anxiety, schizophrenia, neuropathic pain, Alzheimer's disease, Parkinson's syndrome, glioma, lung cancer, liver cancer, breast cancer, colon cancer, rheumatoid arthritis, systemic lupus erythematosus, osteoarthritis, gouty arthritis, skin inflammation, autoimmune diseases, cardiovascular diseases, atherosclerosis, colorectal inflammation, allergic inflammation, hepatitis, and pneumonia.
